(19) United States
(12) Patent Application Publication
(10) Pub. No.: US 2015/0352117 A1
(43) Pub. Date: Dec. 10, 2015

(54) COMBINATION

(71) Applicant: Novartis AG, Basel (CH)

(72) Inventors: Tona M Gilmer, Research Triangle Park, NC (US); Rakesh Kumar, Collegeville, PA (US); Li Liu, King of Prussia, PA (US)

(21) Appl. No.: 14/829,689

(22) Filed: Aug. 19, 2015

Related U.S. Application Data
(63) Continuation of application No. 14/327,660, filed on Jul. 10, 2014.
Application No. 14/327,660 is a continuation of application No. 13/819,091, filed on Feb. 26, 2013, now abandoned.
(60) Provisional application No. 61/377,196, filed on Aug. 26, 2010.

(51) Int. Cl. A61K 31/519; A61K 31/506

(52) U.S. Cl. CPC A61K 31/519; A61K 31/506

(57) ABSTRACT

The present invention relates to a method of treating cancer in a human and to pharmaceutical combinations useful in such treatment. In particular, the method relates to a cancer treatment method that includes administering 5-[[4-[(2,3-dimethyl-2H-indazol-6-yl)methylamino]-2-pyrimidinyl]amino]-2-methylbenzenesulfonamide, or a pharmaceutically acceptable salt thereof, and N-{3-[3-cyclopropyl-5-(2-fluoro-4-iodo-phenylamino)6,8-dimethy;-2,4,7-trioxo-3,4,6,7-tetrahydro-2H-pyrido[4,3-d]pyrimidin-1-yl]phenyl}acetamide, or a pharmaceutically acceptable salt or solvate thereof, to a human in need thereof.

### FIELD OF THE INVENTION

The present invention relates to a method of treating cancer in a mammal and to combinations useful in such treatment. In particular, the method relates to a novel combination comprising the VEGFR inhibitor: 5-[[4-[(2,3-dimethyl-2H-indazol-6-yl)methylamino]-2-pyrimidinyl]amino]-2-methylbenzenesulfonamide, or a pharmaceutically acceptable salt thereof, and MEK inhibitor: N-{3-[3-cyclopropyl-5-(2-fluoro-4-iodo-phenylamino)6,8-dimethy;-2,4,7-trioxo-3,4,6,7-tetrahydro-2H-pyrido[4,3-d]pyrimidin-1-yl]phenyl}acetamide, or a pharmaceutically acceptable salt or solvate thereof, pharmaceutical compositions comprising the same, and methods of using such combinations in the treatment of cancer.

### BACKGROUND OF THE INVENTION

Generally, cancer results from the deregulation of the normal processes that control cell division, differentiation and apoptotic cell death. Apoptosis (programmed cell death) plays essential roles in the embryonic development and pathogenesis of various diseases, such as degenerative neuronal diseases, cardiovascular diseases and cancer. One of the most commonly studied pathways, which involves kinase regulation of apoptosis, is cellular signaling from growth factor receptors at the cell surface to the nucleus (Crews and Erikson, Cell, 74:215-17, 1993).

The process of angiogenesis is the development of new blood vessels from the pre-existing vasculature. Angiogenesis is defined herein as involving: (i) activation of endothelial cells; (ii) increased vascular permeability; (iii) subsequent dissolution of the basement membrane and extravasation of plasma components leading to formation of a provisional fibrin gel extracellular matrix; (iv) proliferation and mobilization of endothelial cells; (v) reorganization of mobilized endothelial cells to form functional capillaries; (vi) capillary loop formation; and (vi) deposition of basement membrane and recruitment of perivascular cells to newly formed vessels. Normal angiogenesis is active during tissue growth from embryonic development through maturity and then enters a period of relative quiescence during adulthood. Normal angiogenesis is also activated during wound healing, and at certain stages of the female reproductive cycle. Inappropriate or pathological angiogenesis has been associated with several disease states including various retinopathies, ischemic disease, atherosclerosis, chronic inflammatory disorders, and cancer. The role of angiogenesis in disease states is discussed, for instance, in Fan et al, Trends in Pharmacol Sci. 16:54-66; Shawveret al, DDT Vol. 2, No. 2 February 1997; Folkmann, 1995, Nature Medicine 1:27-31.

In cancer the growth of solid tumors has been shown to be dependent on angiogenesis. The progression of leukemias as well as the accumulation of fluid associated with malignant ascites and pleural effusions also involve pro-angiogenic factors. (See Folkmann, J., J. Nat'l. Cancer Inst, 1990, 82, 4-6).

Central to the process of angiogenesis are vascular endothelial growth factor (VEGF) and its receptors, termed vascular endothelial growth factor receptor(s) (VEGFRs), The roles VEGF and VEGFRs play in the vascularization of solid tumors, progression of hematopoietic cancers and modulation of vascular permeability have drawn great interest in the scientific community. VEGF is a polypeptide, which has been linked to inappropriate or pathological angiogenesis (Pinedo, H. M. et al The Oncologist, Vol. 5, No. 90001, 1-2, Apr. 2000). VEGFR(s) are protein tyrosine kinases (PTKs) that catalyze the phosphorylation of specific tyrosine residues in proteins that are involved in the regulation of cell growth, differentiation, and survival. (A. F. Wilks, Progress in Growth Factor Research, 1990, 2, 97-111; S. A. Courtneidge, Dev. Supp. 1, 1993, 57-64; J. A.

Cooper, Semin. Cell Biol., 1994, 5(6),377-387; R. F. Paulson, Semin. Immunol. 1995, 7(4), 267-277; A. C. Chan, Curro Opin. Immunol. 1996, 8(3), 394-401).

Three PTK receptors for VEGF have been identified: VEGFR1 (Flt-1); VEGFR2 (Flk-1 and KDR) and VEGFR3 (Flt-4). These receptors are involved in angiogenesis and participate in signal transduction. (Mustonen, T. et al J. Cell. Biol. 1995: 129:895-898; Ferrara and Davis-Smyth, Endocrine Reviews, 18(1):4-25, 1997; McMahon, G., The Oncologist, Vol. 5, No 90001, 3-10, Apr. 2000).

Of particular interest is VEGFR2, which is a transmembrane receptor PTK expressed primarily in endothelial cells. Activation of VEGFR2 by VEGF is a critical step in the signal transduction pathway that initiates tumor angiogenesis. VEGF expression may be constitutive to tumor cells and can also be upregulated in response to certain stimuli. One such stimulus is hypoxia, where VEGF expression is upregulated in both tumor and associated host tissues. The VEGF ligand activates VEGFR2 by binding to its extracellular VEGF binding site. This leads to receptor dimerization of VEGFRs and autophosphorylation of tyrosine residues at the intracellular kinase domain of VEGFR2. The kinase domain operates to transfer a phosphate from ATP to the tyrosine residues, thus providing binding sites for signaling proteins downstream of VEGFR2 leading ultimately to angiogenesis. (Ferrara and Davis-Smyth, Endocrine Reviews, 18(1):4-25, 1997; McMahon, G. The Oncologist, Vol. 5, No. 9000l, 3-10, Apr. 2000.)

Consequently, antagonism of the VEGFR2 kinase domain would block phosphorylation of tyrosine residues and serve to disrupt initiation of angiogenesis. Specifically, inhibition at the ATP binding site of the VEGFR2 kinase domain would prevent binding of ATP and prevent phosphorylation of tyrosine residues. Such disruption of the proangiogenesis signal transduction pathway associated with VEGFR2 should therefore inhibit tumor angiogenesis and thereby provide a potent treatment for cancer or other disorders associated with inappropriate angiogenesis.

Mitogen-activated protein (MAP) Kinase/extracellular signal-regulated kinase (ERK) kinase (hereinafter referred to as MEK) is known to be involved in the regulation of cell proliferation as a kinase that mediates Raf-MEK-ERK signal transduction pathway, and the Raf family (B-Raf, C-Raf etc.) activates the MEK family (MEK-1, MEK-2 etc.) and the MEK family activates the ERK family (ERK-1 and ERK-2).

Activation of Raf-MEK-ERK signal transduction pathway in cancer, particularly colorectal cancer, pancreatic cancer, lung cancer, breast cancer and the like, has been frequently observed.

In addition, since the signals produced by signal molecules such as growth factor, cytokine and the like converge to the activation of MEK-ERK, inhibitors of these functions are considered to more effectively suppress Raf-MEK-ERK signal transduction than suppression of the function of upstream kinases such as RTK, Ras, and Raf.

Moreover, it is also known that a compound having MEK inhibitory activity effectively induces inhibition of ERK1/2 activity and suppression of cell proliferation (The Journal of Biological Chemistry, vol. 276, No. 4, pp. 2686-2692, 2001), and the compound is expected to show effects on diseases caused by undesirable cell proliferation, such as tumor genesis and/or cancer.

It would be useful to provide a novel therapy which provides more effective and/or enhanced treatment of an individual suffering the effects of cancer.

### SUMMARY OF THE INVENTION

One embodiment of this invention provides a combination comprising:

(i) a compound of Structure (I):

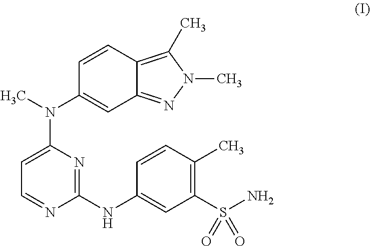 [SMILES: Cc1ccc(Nc2nccc(N(C)c3ccc4c(C)n(C)nc4c3)n2)cc1S(N)(=O)=O]

or a pharmaceutically acceptable salt thereof; and

(ii) a compound of Structure (II):

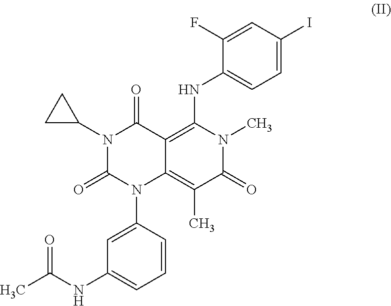 [SMILES: CC(=O)Nc1cccc(-n2c(=O)n(C3CC3)c(=O)c3c(Nc4ccc(I)cc4F)n(C)c(=O)c(C)c32)c1]

or a pharmaceutically acceptable salt or solvate thereof.

One embodiment of this invention provides a method of treating cancer in a human in need thereof which comprises the in vivo administration of a therapeutically effective amount of a combination of 5-[[4-[(2,3-dimethyl-2H-indazol-6-yl)methylamino]-2-pyrimidinyl]amino]-2-methylbenzenesulfonamide, or a pharmaceutically acceptable salt, suitably the monohydrochloride salt, thereof, and N-{3-[3-cyclopropyl-5-(2-fluoro-4-iodo-phenylamino)-6,8-dimethy-2,4,7-trioxo-3,4,6,7-tetrahydro-2H-pyrido[4,3-d]pyrimidin-1-yl]phenyl}acetamide, or a pharmaceutically acceptable salt or solvate, suitably the dimethyl sulfoxide solvate, thereof, to such human.

One embodiment of this invention provides a method of treating cancer in a human in need thereof which comprises the in vivo administration of a therapeutically effective amount of a combination of 5-[[4-[(2,3-dimethyl-2H-indazol-6-yl)methylamino]-2-pyrimidinyl]amino]-2-methylbenzenesulfonamide, or a pharmaceutically acceptable salt, suitably the monohydrochloride salt, thereof, and N-{3-[3-cyclopropyl-5-(2-fluoro-4-iodo-phenylamino)-6,8-dimethy-2,4,7-trioxo-3,4,6, 7-tetrahydro-2H-pyrido[4,3-d]pyrimidin-1-yl]phenyl}acetamide, or a pharmaceutically acceptable salt or solvate, suitably the dimethyl sulfoxide solvate, thereof, to such human,
wherein the combination is administered within a specified period, and
wherein the combination is administered for a duration of time.

One embodiment of this invention provides a method of treating cancer in a human in need thereof which comprises the in vivo administration of a therapeutically effective amount of a combination of 5-[[4-[(2,3-dimethyl-2H-indazol-6-yl)methylamino]-2-pyrimidinyl]amino]-2-methylbenzenesulfonamide, or a pharmaceutically acceptable salt, suitably the monohydrochloride salt, thereof, and N-{3-[3-cyclopropyl-5-(2-fluoro-4-iodo-phenylamino)-6,8-dimethy-2,4,7-trioxo-3,4,6,7-tetrahydro-2H-pyrido[4,3-d]pyrimidin-1-yl]phenyl}acetamide, or a pharmaceutically acceptable salt or solvate, suitably the dimethyl sulfoxide solvate, thereof, to such human,
wherein the compounds of the combination are administered sequentially.

### DETAILED DESCRIPTION OF THE INVENTION

The present invention relates to combinations that exhibit antiproliferative activity. Suitably, the method relates to methods of treating cancer by the co-administration of 5-[[4-[(2,3-dimethyl-2H-indazol-6-yl)methylamino]-2-pyrimidinyl]amino]-2-methylbenzenesulfonamide, or a pharmaceutically acceptable salt, suitably the monohydrochloride salt, thereof, (hereinafter Compound A, or a pharmaceutically acceptable salt, suitably the monohydrochloride salt, thereof,

which compound is represented by Structure I:

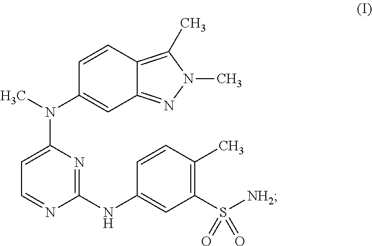 [SMILES: Cc1ccc(Nc2nccc(N(C)c3ccc4c(C)n(C)nc4c3)n2)cc1S(N)(=O)=O]

and N-{3-[3-cyclopropyl-5-(2-fluoro-4-iodo-phenylamino)-6,8-dimethy-2,4,7-trioxo-3,4,6,7-tetrahydro-2H-pyrido[4,3-d]pyrimidin-1-yl]phenyl}acetamide, or a pharmaceutically acceptable salt or solvate, suitably the dimethyl sulfoxide solvate, thereof, (hereinafter Compound B or a pharmaceutically acceptable salt or solvate, suitably the dimethyl sulfoxide solvate, thereof,

which compound is represented by Structure II:

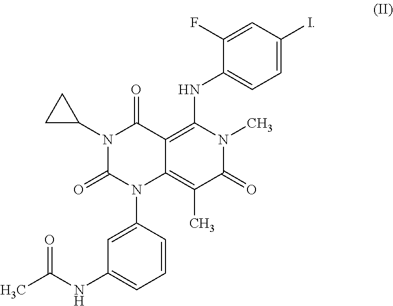 [SMILES: CC(=O)Nc1cccc(-n2c(=O)n(C3CC3)c(=O)c3c(Nc4ccc(I)cc4F)n(C)c(=O)c(C)c32)c1]

Compound A is disclosed and claimed, along with pharmaceutically acceptable salts thereof, as being useful as an inhibitor of VEGFR activity, particularly in treatment of cancer, in International Application No. PCT/US01/49367, having an International filing date of Dec. 19, 2001, International Publication Number WO02/059110 and an International Publication date of Aug. 1, 2002, the entire disclosure of which is hereby incorporated by reference, Compound A is the compound of Example 69. Compound A can be prepared as described in International Application No. PCT/US01/49367.

Suitably, Compound A is in the form of a monohydrochloride salt. This salt form can be prepared by one of skill in the art from the description in International Application No. PCT/US01/49367, having an International filing date of Dec. 19, 2001.

Compound A is sold commercially as the monohydrochloride salt. Compound A is known by the generic name pazopanib and the trade name Votrient®.

Compound B is disclosed and claimed, along with pharmaceutically acceptable salts and solvates thereof, as being useful as an inhibitor of MEK activity, particularly in treatment of cancer, in International Application No. PCT/JP2005/011082, having an International filing date of Jun. 10, 2005; International Publication Number WO 2005/121142 and an International Publication date of Dec. 22, 2005, the entire disclosure of which is hereby incorporated by reference, Compound B is the compound of Example 4-1. Compound B can be prepared as described in International Application No. PCT/JP2005/011082. Compound B can be prepared as described in United States Patent Publication No. US 2006/0014768, Published Jan. 19, 2006, the entire disclosure of which is hereby incorporated by reference.

Suitably, Compound B is in the form of a dimethyl sulfoxide solvate. Suitably, Compound B is in the form of a sodium salt. Suitably, Compound B is in the form of a solvate selected from: hydrate, acetic acid, ethanol, nitromethane, chlorobenzene, 1-pentanci, isopropyl alcohol, ethylene glycol and 3-methyl-1-butanol. These solvates and salt forms can be prepared by one of skill in the art from the description in International Application No. PCT/JP2005/011082 or United States Patent Publication No. US 2006/0014768.

The administration of a therapeutically effective amount of the combinations of the invention are advantageous over the individual component compounds in that the combinations will provide one or more of the following improved properties when compared to the individual administration of a therapeutically effective amount of a component compound: i) a greater anticancer effect than the most active single agent, ii) synergistic or highly synergistic anticancer activity, iii) a dosing protocol that provides enhanced anticancer activity with reduced side effect profile, iv) a reduction in the toxic effect profile, v) an increase in the therapeutic window, or vi) an increase in the bioavailability of one or both of the component compounds.

The compounds of the invention may contain one or more chiral atoms, or may otherwise be capable of existing as two enantiomers. Accordingly, the compounds of this invention include mixtures of enantiomers as well as purified enantiomers or enantiomerically enriched mixtures. Also, it is understood that all tautomers and mixtures of tautomers are included within the scope of Compound A, and pharmaceutically acceptable salts thereof, and Compound B, and pharmaceutically acceptable salts or solvates thereof.

The compounds of the invention may form a solvate which is understood to be a complex of variable stoichiometry formed by a solute (in this invention, Compound A or a salt thereof and/or Compound B or a salt thereof) and a solvent. Such solvents for the purpose of the invention may not interfere with the biological activity of the solute. Examples of suitable solvents include, but are not limited to, water, methanol, dimethyl sulfoxide, ethanol and acetic acid. Suitably the solvent used is a pharmaceutically acceptable solvent. Suitably the solvent used is water or dimethyl sulfoxide.

The pharmaceutically acceptable salts of the compounds of the invention are readily prepared by those of skill in the art.

Also, contemplated herein is a method of treating cancer using a combination of the invention where Compound A, or a pharmaceutically acceptable salt thereof, and/or Compound B or a pharmaceutically acceptable salt or solvate thereof are administered as pro-drugs. Pharmaceutically acceptable pro-drugs of the compounds of the invention are readily prepared by those of skill in the art.

When referring to a dosing protocol, the term “day”, “per day” and the like, refer to a time within one calendar day which begins at midnight and ends at the following midnight.

By the term “treating” and derivatives thereof as used herein, is meant therapeutic therapy. In reference to a particular condition, treating means: (1) to ameliorate or prevent the condition of one or more of the biological manifestations of the condition, (2) to interfere with (a) one or more points in the biological cascade that leads to or is responsible for the condition or (b) one or more of the biological manifestations of the condition, (3) to alleviate one or more of the symptoms, effects or side effects associated with the condition or treatment thereof, or (4) to slow the progression of the condition or one or more of the biological manifestations of the condition. Prophylactic therapy is also contemplated thereby. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof. Prophylactic therapy is appropriate, for example, when a subject is considered at high risk for developing cancer, such as when a subject has a strong family history of cancer or when a subject has been exposed to a carcinogen.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

By the term “combination” and derivatives thereof, as used herein is meant either, simultaneous administration or any manner of separate sequential administration of a therapeutically effective amount of Compound A, or a pharmaceutically acceptable salt thereof, and Compound B or a pharmaceutically acceptable salt or solvate thereof. Preferably, if the administration is not simultaneous, the compounds are administered in a close time proximity to each other. Furthermore, it does not matter if the compounds are administered in the same dosage form, e.g. one compound may be administered topically and the other compound may be administered orally. Suitably, both compounds are administered orally.

By the term “combination kit” as used herein is meant the pharmaceutical composition or compositions that are used to administer Compound A, or a pharmaceutically acceptable salt thereof, and Compound B, or a pharmaceutically acceptable salt or solvate thereof, according to the invention. When both compounds are administered simultaneously, the combination kit can contain Compound A, or a pharmaceutically acceptable salt thereof, and Compound B, or a pharmaceutically acceptable salt or solvate thereof, in a single pharmaceutical composition, such as a tablet, or in separate pharmaceutical compositions. When the compounds are not administered simultaneously, the combination kit will contain Compound A, or a pharmaceutically acceptable salt thereof, and Compound B, or a pharmaceutically acceptable salt or solvate thereof, in separate pharmaceutical compositions. The combination kit can comprise Compound A, or a pharmaceutically acceptable salt thereof, and Compound B, or a pharmaceutically acceptable salt or solvate thereof, in separate pharmaceutical compositions in a single package or in separate pharmaceutical compositions in separate packages.

In one aspect there is provided a combination kit comprising the components:

Compound A, or a pharmaceutically acceptable salt thereof, in association with a pharmaceutically acceptable carrier; and

Compound B, or a pharmaceutically acceptable salt or solvate thereof, in association with a pharmaceutically acceptable carrier.

In one embodiment of the invention the combination kit comprises the following components:

Compound A, or a pharmaceutically acceptable salt thereof, in association with a pharmaceutically acceptable carrier; and

Compound B, or a pharmaceutically acceptable salt or solvate thereof, in association with a pharmaceutically acceptable carrier,

wherein the components are provided in a form which is suitable for sequential, separate and/or simultaneous administration.

In one embodiment the combination kit comprises:

a first container comprising Compound A, or a pharmaceutically acceptable salt thereof, in association with a pharmaceutically acceptable carrier; and

a second container comprising Compound B, or a pharmaceutically acceptable salt or solvate thereof, in association with a pharmaceutically acceptable carrier, and a container means for containing said first and second containers.

The “combination kit” can also be provided by instruction, such as dosage and administration instructions. Such dosage and administration instructions can be of the kind that is provided to a doctor, for example by a drug product label, or they can be of the kind that is provided by a doctor, such as instructions to a patient.

Unless otherwise defined, in all dosing protocols described herein, the regimen of compounds administered does not have to commence with the start of treatment and terminate with the end of treatment, it is only required that the number of consecutive days in which both compounds are administered and the optional number of consecutive days in which only one of the component compounds is administered, or the indicated dosing protocol—including the amount of compound administered, occur at some point during the course of treatment.

As used herein the term “Compound A²” means - - - Compound A, or a pharmaceutically acceptable salt thereof - - - .

As used herein the term “Compound B²” means - - - Compound B, or a pharmaceutically acceptable salt or solvate thereof - - - .

The term “loading dose” as used herein will be understood to mean a single dose or short duration regimen of Compound A or Compound B having a dosage higher than the maintenance dose administered to the subject to rapidly increase the blood concentration level of the drug. Suitably, a short duration regimen for use herein will be from: 1 to 14 days; suitably from 1 to 7 days; suitably from 1 to 3 days; suitably for three days; suitably for two days; suitably for one day. In some embodiments, the “loading dose” can increase the blood concentration of the drug to a therapeutically effective level. In some embodiments, the “loading dose” can increase the blood concentration of the drug to a therapeutically effective level in conjunction with a maintenance dose of the drug. The “loading dose” can be administered once per day, or more than once per day (e.g., up to 4 times per day). Suitably the “loading dose” will be administered once a day. Suitably, the loading dose will be an amount from 2 to 100 times the maintenance dose; suitably from 2 to 10 times; suitably from 2 to 5 times; suitably 2 times; suitably 3 times; suitably 4 times; suitably 5 times. Suitably, the loading dose will be administered for from 1 to 7 days; suitably from 1 to 5 days; suitably from 1 to 3 days; suitably for 1 day; suitably for 2 days; suitably for 3 days, followed by a maintenance dosing protocol.

The term “maintenance dose” as used herein will be understood to mean a dose that is serially administered (for example., at least twice), and which is intended to either slowly raise blood concentration levels of the compound to a therapeutically effective level, or to maintain such a therapeutically effective level. The maintenance dose is generally administered once per day and the daily dose of the maintenance dose is lower than the total daily dose of the loading dose.

Suitably the combinations of this invention are administered within a “specified period”.

By the term “specified period” and derivatives thereof, as used herein is meant the interval of time between the administration of one of Compound A² and Compound B² and the other of Compound A² and Compound B². Unless otherwise defined, the specified period can include simultaneous administration. When both compounds of the invention are administered once a day the specified period refers to timing of the administration of Compound A² and Compound B² during a single day. When one or both compounds of the invention are administered more than once a day, the specified period is calculated based on the first administration of each compound on a specific day. All administrations of a compound of the invention that are subsequent to the first during a specific day are not considered when calculating the specific period.

Suitably, if the compounds are administered within a “specified period” and not administered simultaneously, they are both administered within about 24 hours of each other—in this case, the specified period will be about 24 hours; suitably they will both be administered within about 12 hours of each other—in this case, the specified period will be about 12 hours; suitably they will both be administered within about 11 hours of each other—in this case, the specified period will be about 11 hours; suitably they will both be administered within about 10 hours of each other—in this case, the specified period will be about 10 hours; suitably they will both be administered within about 9 hours of each other—in this case, the specified period will be about 9 hours; suitably they will both be administered within about 8 hours of each other—in this case, the specified period will be about 8 hours; suitably they will both be administered within about 7 hours of each other—in this case, the specified period will be about 7 hours; suitably they will both be administered within about 6 hours of each other—in this case, the specified period will be about 6 hours; suitably they will both be administered within about 5 hours of each other—in this case, the specified period will be about 5 hours; suitably they will both be administered within about 4 hours of each other—in this case, the specified period will be about 4 hours; suitably they will both be administered within about 3 hours of each other—in this case, the specified period will be about 3 hours; suitably they will be administered within about 2 hours of each other—in this case, the specified period will be about 2 hours; suitably they will both be administered within about 1 hour of each other—in this case, the specified period will be about 1 hour. As used herein, the administration of Compound A² and Compound B² in less than about 45 minutes apart is considered simultaneous administration.

Suitably, when the combination of the invention is administered for a “specified period”, the compounds will be co-administered for a “duration of time”.

By the term “duration of time” and derivatives thereof, as used herein is meant that both compounds of the invention are administered within a “specified period” for an indicated number of consecutive days, optionally followed by a number of consecutive days where only one of the component compounds is administered.

Regarding “specified period” administration:

Suitably, during the course of treatment, both compounds will be administered within a specified period for at least 1 day—in this case, the duration of time will be at least 1 day; suitably, during the course of treatment, both compounds will be administered within a specified period for at least 2 consecutive days—in this case, the duration of time will be at least 2 days; suitably, during the course of treatment, both compounds will be administered within a specified period for at least 3 consecutive days—in this case, the duration of time will be at least 3 days; suitably, during the course of treatment, both compounds will be administered within a specified period for at least 5 consecutive days—in this case, the duration of time will be at least 5 days; suitably, during the course of treatment, both compounds will be administered within a specified period for at least 7 consecutive days—in this case, the duration of time will be at least 7 days; suitably, during the course of treatment, both compounds will be administered within a specified period for at least 14 consecutive days—in this case, the duration of time will be at least 14 days; suitably, during the course of treatment, both compounds will be administered within a specified period for at least 30 consecutive days—in this case, the duration of time will be at least 30 days. When, during the course of treatment, both compounds are administered within a specified period for over 30 days, the treatment is considered chronic treatment and will continue until an altering event, such as a reassessment in cancer status or a change in the condition of the patient, warrants a modification to the protocol.

Further regarding “specified period” administration:

Suitably, during the course of treatment, both compounds will be administered within a specified period for at least 1 day, followed by the administration of Compound A² alone for at least 1 day—in this case, the duration of time will be at least 2 days;

suitably, during the course of treatment, both compounds will be administered within a specified period for at least 1 day, followed by administration of Compound A² alone for at least 2 days—in this case, the duration of time will be at least 3 days; suitably, during the course of treatment, both compounds will be administered within a specified period for at least 1 day, followed by administration of Compound A² alone for at least 3 days—in this case, the duration of time will be at least 4 days; suitably, during the course of treatment, both compounds will be administered within a specified period for at least 1 day, followed by administration of Compound A² alone for at least 4 days—in this case, the duration of time will be at least 5 days; suitably, during the course of treatment, both compounds will be administered within a specified period for at least 1 day, followed by administration of Compound A² alone for at least 5 days—in this case, the duration of time will be at least 6 days; suitably, during the course of treatment, both compounds will be administered within a specified period for at least 1 day, followed by administration of Compound A² alone for at least 6 days—in this case, the duration of time will be at least 7 days; suitably, during the course of treatment, both compounds will be administered within a specified period for at least 1 day, followed by administration of Compound A² alone for at least 7 days—in this case, the duration of time will be at least 8 days; suitably, during the course of treatment, both compounds will be administered within a specified period for at least 2 consecutive days, followed by administration of Compound A² alone for at least 1 day—in this case, the duration of time will be at least 3 days; suitably, during the course of treatment, both compounds will be administered within a specified period for at least 2 consecutive days, followed by administration of Compound A² alone for at least 2 consecutive days—in this case, the duration of time will be at least 4 days; suitably, during the course of treatment, both compounds will be administered within a specified period for at least 2 consecutive days, followed by administration of Compound A² alone for at least 3 consecutive days—in this case, the duration of time will be at least 5 days; suitably, during the course of treatment, both compounds will be administered within a specified period for at least 2 consecutive days, followed by administration of Compound A² alone for at least 4 consecutive days—in this case, the duration of time will be at least 6 days; suitably, during the course of treatment, both compounds will be administered within a specified period for at least 2 consecutive days, followed by administration of Compound A² alone for at least 5 consecutive days—in this case, the duration of time will be at least 7 days; suitably, during the course of treatment, both compounds will be administered within a specified period for at least 2 consecutive days, followed by administration of Compound A² alone for at least 6 consecutive days—in this case, the duration of time will be at least 8 days; suitably, during the course of treatment, both compounds will be administered within a specified period for at least 2 consecutive days, followed by administration of Compound A² alone for at least 7 consecutive days—in this case, the duration of time will be at least 9 days; suitably, during the course of treatment, both compounds will be administered within a specified period for at least 3 consecutive days, followed by administration of Compound A² alone for at least 1 day—in this case, the duration of time will be at least 4 days; suitably, during the course of treatment, both compounds will be administered within a specified period for at least 3 consecutive days, followed by administration of Compound A² alone for at least 2 consecutive days—in this case, the duration of time will be at least 5 days; suitably, during the course of treatment, both compounds will be administered within a specified period for at least 3 consecutive days, followed by administration of Compound A² alone for at least 3 consecutive days—in this case, the duration of time will be at least 6 days; suitably, during the course of treatment, both compounds will be administered within a specified period for at least 3 consecutive days, followed by administration of Compound A² alone for at least 4 consecutive days—in this case, the duration of time will be at least 7 days; suitably, during the course of treatment, both compounds will be administered within a specified period for at least 3 consecutive days, followed by administration of Compound A² alone for at least 5 consecutive days—in this case, the duration of time will be at least 8 days; suitably, during the course of treatment, both compounds will be administered within a specified period for at least 3 consecutive days, followed by administration of Compound A² alone for at least 6 consecutive days—in this case, the duration of time will be at least 9 days; suitably, during the course of treatment, both compounds will be administered within a specified period for at least 3 consecutive days, followed by administration of Compound A² alone for at least 7 consecutive days—in this case, the duration of time will be at least 10 days; suitably, during the course of treatment, both compounds will be administered within a specified period for at least 4 consecutive days, followed by administration of Compound A² alone for at least 1 day—in this case, the duration of time will be at least 5 consecutive days; suitably, during the course of treatment, both compounds will be administered within a specified period for at least 4 consecutive days, followed by administration of Compound A² alone for at least 2 consecutive days—in this case, the duration of time will be at least 6 consecutive days; suitably, during the course of treatment, both compounds will be administered within a specified period for at least 4 consecutive days, followed by administration of Compound A² alone for at least 3 consecutive days—in this case, the duration of time will be at least 7 consecutive days; suitably, during the course of treatment, both compounds will be administered within a specified period for at least 4 consecutive days, followed by administration of Compound A² alone for at least 4 consecutive days—in this case, the duration of time will be at least 8 consecutive days; suitably, during the course of treatment, both compounds will be administered within a specified period for at least 4 consecutive days, followed by administration of Compound A² alone for at least 7 consecutive days—in this case, the duration of time will be at least 11 consecutive days; suitably, during the course of treatment, both compounds will be administered within a specified period for at least 5 consecutive days, followed by administration of Compound A² alone for at least 1 day—in this case, the duration of time will be at least 6 consecutive days; suitably, during the course of treatment, both compounds will be administered within a specified period for at least 5 consecutive days, followed by administration of Compound A² alone for at least 2 consecutive days—in this case, the duration of time will be at least 7 consecutive days; suitably, during the course of treatment, both compounds will be administered within a specified period for at least 5 consecutive days, followed by administration of Compound A² alone for at least 3 consecutive days—in this case, the duration of time will be at least 8 consecutive days; suitably, during the course of treatment, both compounds will be administered within a specified period for at least 5 consecutive days, followed by administration of Compound A² alone for at least 4 consecutive days—in this case, the duration of time will be at least 9 consecutive days; suitably, during the course of treatment, both compounds will be administered within a specified period for at least 5 consecutive days, followed by administration of Compound A² alone for at least 5 consecutive days—in this case, the duration of time will be at least 10 consecutive days; suitably, during the course of treatment, both compounds will be administered within a specified period for at least 7 consecutive days, followed by administration of Compound A² alone for at least 2 consecutive days—in this case, the duration of time will be at least 9 consecutive days; suitably, during the course of treatment, both compounds will be administered within a specified period for at least 14 consecutive days, followed by administration of Compound A² alone for at least 7 consecutive days—in this case, the duration of time will be at least 21 consecutive days; suitably, during the course of treatment, both compounds will be administered within a specified period for at least 30 consecutive days, followed by administration of Compound A² alone for at least 7 consecutive days—in this case, the duration of time will be at least 37 consecutive days. Suitably, during the course of treatment, both compounds will be administered within a specified period for from 1 to 3 consecutive days, followed by administration of Compound A² alone for from 3 to 7 consecutive days. Suitably, during the course of treatment, both compounds will be administered within a specified period for from 3 to 6 consecutive days, followed by administration of Compound A² alone for from 1 to 4 consecutive days. Suitably, during the course of treatment, both compounds will be administered within a specified period for 5 consecutive days, followed by administration of Compound A² alone for 2 consecutive days. Suitably, during the course of treatment, both compounds will be administered within a specified period for 2 consecutive days, followed by administration of Compound A² alone for from 3 to 7 consecutive days. Suitably, during the course of treatment, both compounds will be administered within a specified period for from 1 to 3 days over a 7 day period, and during the other days of the 7 day period Compound A² will be administered alone. Suitably, during the course of treatment, both compounds will be administered within a specified period for 2 days over a 7 day period, and during the other days of the 7 day period Compound A² will be administered alone.

Further regarding “specified period” administration:

Suitably, during the course of treatment, both compounds will be administered within a specified period for at least 1 day, followed by the administration of Compound B² alone for at least 1 day—in this case, the duration of time will be at least 2 days; suitably, during the course of treatment, both compounds will be administered within a specified period for at least 1 day, followed by administration of Compound B² alone for at least 2 days—in this case, the duration of time will be at least 3 days; suitably, during the course of treatment, both compounds will be administered within a specified period for at least 1 day, followed by administration of Compound B² alone for at least 3 days—in this case, the duration of time will be at least 4 days; suitably, during the course of treatment, both compounds will be administered within a specified period for at least 1 day, followed by administration of Compound B² alone for at least 4 days—in this case, the duration of time will be at least 5 days; suitably, during the course of treatment, both compounds will be administered within a specified period for at least 1 day, followed by administration of Compound B² alone for at least 5 days—in this case, the duration of time will be at least 6 days; suitably, during the course of treatment, both compounds will be administered within a specified period for at least 1 day, followed by administration of Compound B² alone for at least 6 days—in this case, the duration of time will be at least 7 days; suitably, during the course of treatment, both compounds will be administered within a specified period for at least 1 day, followed by administration of Compound B² alone for at least 7 days—in this case, the duration of time will be at least 8 days; suitably, during the course of treatment, both compounds will be administered within a specified period for at least 2 consecutive days, followed by administration of Compound B² alone for at least 1 day—in this case, the duration of time will be at least 3 days; suitably, during the course of treatment, both compounds will be administered within a specified period for at least 2 consecutive days, followed by administration of Compound B² alone for at least 2 consecutive days—in this case, the duration of time will be at least 4 days; suitably, during the course of treatment, both compounds will be administered within a specified period for at least 2 consecutive days, followed by administration of Compound B² alone for at least 3 consecutive days—in this case, the duration of time will be at least 5 days; suitably, during the course of treatment, both compounds will be administered within a specified period for at least 2 consecutive days, followed by administration of Compound B² alone for at least 4 consecutive days—in this case, the duration of time will be at least 6 days; suitably, during the course of treatment, both compounds will be administered within a specified period for at least 2 consecutive days, followed by administration of Compound B² alone for at least 5 consecutive days—in this case, the duration of time will be at least 7 days; suitably, during the course of treatment, both compounds will be administered within a specified period for at least 2 consecutive days, followed by administration of Compound B² alone for at least 6 consecutive days—in this case, the duration of time will be at least 8 days; suitably, during the course of treatment, both compounds will be administered within a specified period for at least 2 consecutive days, followed by administration of Compound B² alone for at least 7 consecutive days—in this case, the duration of time will be at least 9 days; suitably, during the course of treatment, both compounds will be administered within a specified period for at least 3 consecutive days, followed by administration of Compound B² alone for at least 1 day—in this case, the duration of time will be at least 4 days; suitably, during the course of treatment, both compounds will be administered within a specified period for at least 3 consecutive days, followed by administration of Compound B² alone for at least 2 consecutive days—in this case, the duration of time will be at least 5 days; suitably, during the course of treatment, both compounds will be administered within a specified period for at least 3 consecutive days, followed by administration of Compound B² alone for at least 3 consecutive days—in this case, the duration of time will be at least 6 days; suitably, during the course of treatment, both compounds will be administered within a specified period for at least 3 consecutive days, followed by administration of Compound B² alone for at least 4 consecutive days—in this case, the duration of time will be at least 7 days; suitably, during the course of treatment, both compounds will be administered within a specified period for at least 3 consecutive days, followed by administration of Compound B² alone for at least 5 consecutive days—in this case, the duration of time will be at least 8 days; suitably, during the course of treatment, both compounds will be administered within a specified period for at least 3 consecutive days, followed by administration of Compound B² alone for at least 6 consecutive days—in this case, the duration of time will be at least 9 days; suitably, during the course of treatment, both compounds will be administered within a specified period for at least 3 consecutive days, followed by administration of Compound B² alone for at least 7 consecutive days—in this case, the duration of time will be at least 10 days; suitably, during the course of treatment, both compounds will be administered within a specified period for at least 4 consecutive days, followed by administration of Compound B² alone for at least 1 day—in this case, the duration of time will be at least 5 consecutive days; suitably, during the course of treatment, both compounds will be administered within a specified period for at least 4 consecutive days, followed by administration of Compound B² alone for at least 2 consecutive days—in this case, the duration of time will be at least 6 consecutive days; suitably, during the course of treatment, both compounds will be administered within a specified period for at least 4 consecutive days, followed by administration of Compound B² alone for at least 3 consecutive days—in this case, the duration of time will be at least 7 consecutive days; suitably, during the course of treatment, both compounds will be administered within a specified period for at least 4 consecutive days, followed by administration of Compound B² alone for at least 4 consecutive days—in this case, the duration of time will be at least 8 consecutive days; suitably, during the course of treatment, both compounds will be administered within a specified period for at least 4 consecutive days, followed by administration of Compound B² alone for at least 7 consecutive days—in this case, the duration of time will be at least 11 consecutive days; suitably, during the course of treatment, both compounds will be administered within a specified period for at least 5 consecutive days, followed by administration of Compound B² alone for at least 1 day—in this case, the duration of time will be at least 6 consecutive days; suitably, during the course of treatment, both compounds will be administered within a specified period for at least 5 consecutive days, followed by administration of Compound B² alone for at least 2 consecutive days—in this case, the duration of time will be at least 7 consecutive days; suitably, during the course of treatment, both compounds will be administered within a specified period for at least 5 consecutive days, followed by administration of Compound B² alone for at least 3 consecutive days—in this case, the duration of time will be at least 8 consecutive days; suitably, during the course of treatment, both compounds will be administered within a specified period for at least 5 consecutive days, followed by administration of Compound B² alone for at least 4 consecutive days—in this case, the duration of time will be at least 9 consecutive days; suitably, during the course of treatment, both compounds will be administered within a specified period for at least 5 consecutive days, followed by administration of Compound B² alone for at least 5 consecutive days—in this case, the duration of time will be at least 10 consecutive days; suitably, during the course of treatment, both compounds will be administered within a specified period for at least 7 consecutive days, followed by administration of Compound B² alone for at least 2 consecutive days—in this case, the duration of time will be at least 9 consecutive days; suitably, during the course of treatment, both compounds will be administered within a specified period for at least 14 consecutive days, followed by administration of Compound B² alone for at least 7 consecutive days—in this case, the duration of time will be at least 21 consecutive days; suitably, during the course of treatment, both compounds will be administered within a specified period for at least 30 consecutive days, followed by administration of Compound B² alone for at least 7 consecutive days—in this case, the duration of time will be at least 37 consecutive days. Suitably, during the course of treatment, both compounds will be administered within a specified period for from 1 to 3 consecutive days, followed by administration of Compound B² alone for from 3 to 7 consecutive days. Suitably, during the course of treatment, both compounds will be administered within a specified period for from 3 to 6 consecutive days, followed by administration of Compound B² alone for from 1 to 4 consecutive days. Suitably, during the course of treatment, both compounds will be administered within a specified period for 5 consecutive days, followed by administration of Compound B² alone for 2 consecutive days. Suitably, during the course of treatment, both compounds will be administered within a specified period for 2 consecutive days, followed by administration of Compound B² alone for from 3 to 7 consecutive days. Suitably, during the course of treatment, both compounds will be administered within a specified period for from 1 to 3 days over a 7 day period, and during the other days of the 7 day period Compound B² will be administered alone. Suitably, during the course of treatment, both compounds will be administered within a specified period for 2 days over a 7 day period, and during the other days of the 7 day period Compound B² will be administered alone.

Further regarding “specified period” administration:

Suitably, during the course of treatment, Compound A² and Compound B² will be administered within a specified period for from 1 to 3 days over a 7 day period, and during the other days of the 7 day period Compound A² will be administered alone. Suitably, this 7 day protocol is repeated for 2 cycles or for 14 days; suitably for 4 cycles or 28 days; suitably for continuous administration.

Suitably, during the course of treatment, Compound A² and Compound B² will be administered within a specified period for from 1 to 3 days over a 7 day period, and during the other days of the 7 day period Compound B² will be administered alone. Suitably, this 7 day protocol is repeated for 2 cycles or for 14 days; suitably for 4 cycles or 28 days; suitably for continuous administration.

Suitably, during the course of treatment, Compound A² and Compound B² will be administered within a specified period for 3 days over a 7 day period, and during the other days of the 7 day period Compound A² will be administered alone. Suitably, this 7 day protocol is repeated for 2 cycles or for 14 days; suitably for 4 cycles or 28 days; suitably for continuous administration.

Suitably, during the course of treatment, Compound A² and Compound B² will be administered within a specified period for 3 days over a 7 day period, and during the other days of the 7 day period Compound B² will be administered alone. Suitably, this 7 day protocol is repeated for 2 cycles or for 14 days; suitably for 4 cycles or 28 days; suitably for continuous administration.

Suitably, during the course of treatment, Compound A² and Compound B² will be administered within a specified period for 2 days over a 7 day period, and during the other days of the 7 day period Compound A² will be administered alone. Suitably, this 7 day protocol is repeated for 2 cycles or for 14 days; suitably for 4 cycles or 28 days; suitably for continuous administration.

Suitably, during the course of treatment, Compound A² and Compound B² will be administered within a specified period for 2 days over a 7 day period, and during the other days of the 7 day period Compound B² will be administered alone. Suitably, this 7 day protocol is repeated for 2 cycles or for 14 days; suitably for 4 cycles or 28 days; suitably for continuous administration.

Suitably, during the course of treatment, Compound A² and Compound B² will be administered within a specified period for 1 day during a 7 day period, and during the other days of the 7 day period Compound A² will be administered alone. Suitably, this 7 day protocol is repeated for 2 cycles or for 14 days; suitably for 4 cycles or 28 days; suitably for continuous administration.

Suitably, during the course of treatment, Compound A² and Compound B² will be administered within a specified period for 1 day during a 7 day period, and during the other days of the 7 day period Compound B² will be administered alone. Suitably, this 7 day protocol is repeated for 2 cycles or for 14 days; suitably for 4 cycles or 28 days; suitably for continuous administration.

Suitably, during the course of treatment, Compound A² and Compound B² will be administered within a specified period for from 1 to 5 days over a 14 day period, and during the other days of the 14 day period Compound A² will be administered alone. Suitably, this 14 day protocol is repeated for 2 cycles or for 28 days; suitably for continuous administration.

Suitably, during the course of treatment, Compound A² and Compound B² will be administered within a specified period for from 1 to 5 days over a 14 day period, and during the other days of the 14 day period Compound B² will be administered alone. Suitably, this 14 day protocol is repeated for 2 cycles or for 28 days; suitably for continuous administration.

Suitably, if the compounds are not administered during a “specified period”, they are administered sequentially. By the term “sequential administration”, and derivates thereof, as used herein is meant that one of Compound A² and Compound B² is administered for 1 or more consecutive days and the other of Compound A² and Compound B² is subsequently administered for 1 or more consecutive days. Unless otherwise defined, the “sequential administration” and in all dosing protocols described herein, do not have to commence with the start of treatment and terminate with the end of treatment, it is only required that the administration of one of Compound A² and Compound B² followed by the administration of the other of Compound A² and Compound B², or the indicated dosing protocol, occur at some point during the course of treatment. Also, contemplated herein is a drug holiday utilized between the sequential administration of one of Compound A² and Compound B² and the other of Compound A² and Compound B². As used herein, a drug holiday is a period of days after the sequential administration of one of Compound A² and Compound B² and before the administration of the other of Compound A² and Compound B² where neither Compound A² nor Compound B² is administered. Suitably the drug holiday will be a period of days selected from: 1 day, 2 days, 3 days, 4 days, 5 days, 6 days, 7 days, 8 days, 9 days, 10 days, 11 days, 12 days, 13 days and 14 days.

Regarding sequential administration:

Suitably, one of Compound A² and Compound B² is administered for from 1 to 30 consecutive days, followed by an optional drug holiday, followed by administration of the other of Compound A² and Compound B² for from 1 to 30 consecutive days. Suitably, one of Compound A² and Compound B² is administered for from 1 to 21 consecutive days, followed by an optional drug holiday, followed by administration of the other of Compound A² and Compound B² for from 1 to 21 consecutive days. Suitably, one of Compound A² and Compound B² is administered for from 1 to 14 consecutive days, followed by a drug holiday of from 1 to 14 days, followed by administration of the other of Compound A² and Compound B² for from 1 to 14 consecutive days. Suitably, one of Compound A² and Compound B² is administered for from 2 to 7 consecutive days, followed by a drug holiday of from 2 to 10 days, followed by administration of the other of Compound A² and Compound B² for from 2 to 7 consecutive days.

Suitably, Compound B² will be administered first in the sequence, followed by an optional drug holiday, followed by administration of Compound A². Suitably, Compound B² is administered for from 1 to 21 consecutive days, followed by an optional drug holiday, followed by administration of Compound A² for from 1 to 21 consecutive days. Suitably, Compound B² is administered for from 3 to 21 consecutive days, followed by a drug holiday of from 1 to 14 days, followed by administration of Compound A² for from 3 to 21 consecutive days. Suitably, Compound B² is administered for from 3 to 21 consecutive days, followed by a drug holiday of from 3 to 14 days, followed by administration of Compound A² for from 3 to 21 consecutive days. Suitably, Compound B² is administered for 21 consecutive days, followed by an optional drug holiday, followed by administration of Compound A² for 14 consecutive days. Suitably, Compound B² is administered for 14 consecutive days, followed by a drug holiday of from 1 to 14 days, followed by administration of Compound A² for 14 consecutive days. Suitably, Compound B² is administered for 7 consecutive days, followed by a drug holiday of from 3 to 10 days, followed by administration of Compound A² for 7 consecutive days. Suitably, Compound B² is administered for 3 consecutive days, followed by a drug holiday of from 3 to 14 days, followed by administration of Compound A² for 7 consecutive days. Suitably, Compound B² is administered for 3 consecutive days, followed by a drug holiday of from 3 to 10 days, followed by administration of Compound A² for 3 consecutive days.

Suitably, Compound A² will be administered first in the sequence, followed by an optional drug holiday, followed by administration of Compound B². Suitably, Compound A² is administered for from 1 to 21 consecutive days, followed by an optional drug holiday, followed by administration of Compound B² for from 1 to 21 consecutive days. Suitably, Compound A² is administered for from 3 to 21 consecutive days, followed by a drug holiday of from 1 to 14 days, followed by administration of Compound B² for from 3 to 21 consecutive days. Suitably, Compound A² is administered for from 3 to 21 consecutive days, followed by a drug holiday of from 3 to 14 days, followed by administration of Compound B² for from 3 to 21 consecutive days. Suitably, Compound A² is administered for 21 consecutive days, followed by an optional drug holiday, followed by administration of Compound B² for 14 consecutive days. Suitably, Compound A² is administered for 14 consecutive days, followed by a drug holiday of from 1 to 14 days, followed by administration of Compound B² for 14 consecutive days. Suitably, Compound A² is administered for 7 consecutive days, followed by a drug holiday of from 3 to 10 days, followed by administration of Compound B² for 7 consecutive days. Suitably, Compound A² is administered for 3 consecutive days, followed by a drug holiday of from 3 to 14 days, followed by administration of Compound B² for 7 consecutive days. Suitably, Compound A² is administered for 3 consecutive days, followed by a drug holiday of from 3 to 10 days, followed by administration of Compound B² for 3 consecutive days. Suitably, Compound A² is administered for 7 consecutive days, followed by administration of Compound B² for 1 day. Suitably, Compound A² is administered for 6 consecutive days, followed by administration of Compound B² for 1 day. Suitably, Compound B² is administered for 1 day, followed by administration of Compound A² for 7 consecutive days. Suitably, Compound B² is administered for 1 day, followed by administration of Compound A² for 6 consecutive days.

It is understood that a “specified period” administration and a “sequential” administration can be followed by repeat dosing or can be followed by an alternate dosing protocol, and a drug holiday may precede the repeat dosing or alternate dosing protocol.

Suitably, the amount of Compound A² administered as part of the combination according to the present invention will be an amount selected from about 50 mg to about 1,200 mg; suitably, the amount will be selected from about 100 mg to about 1,000 mg; suitably, the amount will be selected from about 100 mg to about 800 mg; suitably, the amount will be selected from about 100 mg to about 600 mg; suitably, the amount will be 50 mg, suitably, the amount will be 100 mg, suitably, the amount will be 200 mg, suitably, the amount will be 400 mg, suitably, the amount will be 600 mg; suitably, the amount will be 800 mg; suitably, the amount will be 1,000 mg; suitably, the amount will be 1,200 mg. Accordingly, the amount of Compound A² administered as part of the combination according to the present invention will be an amount selected from about 50 mg to about 1,200 mg. For example, the amount of Compound A² administered as part of the combination according to the present invention is suitably selected from 50 mg, 100 mg, 200 mg, 400 mg, 600 mg, 800 mg, 1,000 mg and 1,200 mg. Suitably, the selected amount of Compound A² is administered from 1 to 4 times a day, in one or more tablets. Suitably, the selected amount of Compound A² is administered twice a day, in one or more tablets. Suitably, the selected amount of Compound A² is administered once a day, in one or more tablets.

Suitably, the amount of Compound B² administered as part of the combination according to the present invention will be an amount selected from about 0.125 mg to about 10 mg; suitably, the amount will be selected from about 0.25 mg to about 9 mg; suitably, the amount will be selected from about 0.25 mg to about 8 mg; suitably, the amount will be selected from about 0.5 mg to about 8 mg; suitably, the amount will be selected from about 0.5 mg to about 7 mg; suitably, the amount will be selected from about 1 mg to about 7 mg; suitably, the amount will be about 5 mg. Accordingly, the amount of Compound A administered as part of the combination according to the present invention will be an amount selected from about 0.125 mg to about 10 mg. For example, the amount of Compound B² administered as part of the combination according to the present invention can be 0.125 mg, 0.25 mg, 0.5 mg, 0.75 mg, 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg.

As used herein, all amounts specified for Compound A² and Compound B² are indicated as the administered amount of free or unsalted compound per dose.

The method of the present invention may also be employed with other therapeutic methods of cancer treatment.

While it is possible that, for use in therapy, therapeutically effective amounts of the combinations of the present invention may be administered as the raw chemical, it is preferable to present the combinations as a pharmaceutical composition or compositions. Accordingly, the invention further provides pharmaceutical compositions, which include Compound A² and/or Compound B², and one or more pharmaceutically acceptable carriers. The combinations of the present invention are as described above. The carrier(s) must be acceptable in the sense of being compatible with the other ingredients of the formulation, capable of pharmaceutical formulation, and not deleterious to the recipient thereof. In accordance with another aspect of the invention there is also provided a process for the preparation of a pharmaceutical formulation including admixing Compound A² and/or Compound B² with one or more pharmaceutically acceptable carriers. As indicated above, such elements of the pharmaceutical combination utilized may be presented in separate pharmaceutical compositions or formulated together in one pharmaceutical formulation.

Pharmaceutical formulations may be presented in unit dose forms containing a predetermined amount of active ingredient per unit dose. As is known to those skilled in the art, the amount of active ingredient per dose will depend on the condition being treated, the route of administration and the age, weight and condition of the patient. Preferred unit dosage formulations are those containing a daily dose or sub-dose, or an appropriate fraction thereof, of an active ingredient. Furthermore, such pharmaceutical formulations may be prepared by any of the methods well known in the pharmacy art.

Compound A² and Compound B² may be administered by any appropriate route. Suitable routes include oral, rectal, nasal, topical (including buccal and sublingual), vaginal, and parenteral (including subcutaneous, intramuscular, intravenous, intradermal, intrathecal, and epidural). It will be appreciated that the preferred route may vary with, for example, the condition of the recipient of the combination and the cancer to be treated. It will also be appreciated that each of the agents administered may be administered by the same or different routes and that Compound A² and Compound B² may be compounded together in a pharmaceutical composition/formulation. Suitably, Compound A² and Compound B² are administered in separate pharmaceutical compositions.

The compounds or combinations of the current invention are incorporated into convenient dosage forms such as capsules, tablets, or injectable preparations. Solid or liquid pharmaceutical carriers are employed. Solid carriers include, starch, lactose, calcium sulfate dihydrate, terra alba, sucrose, talc, gelatin, agar, pectin, acacia, magnesium stearate, and stearic acid. Liquid carriers include syrup, peanut oil, olive oil, saline, and water. Similarly, the carrier may include a prolonged release material, such as glyceryl monostearate or glyceryl distearate, alone or with a wax. The amount of solid carrier varies widely but, suitably, may be from about 25 mg to about 1 g per dosage unit. When a liquid carrier is used, the preparation will suitably be in the form of a syrup, elixir, emulsion, soft gelatin capsule, sterile injectable liquid such as an ampoule, or an aqueous or nonaqueous liquid suspension.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

It should be understood that in addition to the ingredients mentioned above, the formulations may include other agents conventional in the art having regard to the type of formulation in question, for example those suitable for oral administration may include flavoring agents.

As indicated, therapeutically effective amounts of the combinations of the invention (Compound A² in combination with Compound B²) are administered to a human. Typically, the therapeutically effective amount of the administered agents of the present invention will depend upon a number of factors including, for example, the age and weight of the subject, the precise condition requiring treatment, the severity of the condition, the nature of the formulation, and the route of administration. Ultimately, the therapeutically effective amount will be at the discretion of the attending physician.

The combinations of the invention are tested for efficacy, advantageous and synergistic properties generally according to known procedures.

Suitably, the combinations of the invention are tested for efficacy, advantageous and synergistic properties generally according to the following combination assays.

### Methods

### Experimental Preparation(s)

### Animals

Female CD-1 nude mice of 6-8 weeks in age were used in these studies; all mice were obtained from Charles River Laboratories (Wilmington, Del.). Animals were housed in pathogen free conditions and handled with aseptic technique.

### Cell Culture

The A375P F11s cell line, encoding a mutation for BRAF^{V600E}, was subcloned from the A375P human melanoma cell line (obtained from ATCC, Cat #CRL-1619). The selected clone (A375P F11s) was isolated and mutation in BRAF (T1799A) encoding the V600E amino acid change was reconfirmed.

The A375P F11s cell lines were grown in RPMI growth medium supplemented with 10% fetal bovine serum (FBS), 1% sodium pyruvate and 1% penicillin-streptomycin in a humidified 37° C. chamber with 5% CO₂. Cells were expanded to obtain the large numbers of cells required for each injection; cells were grown in log phase, without reaching confluency and passaged regularly.

### Establishing Xenograft Tumors from Cell Suspension

A375P F11s cells were harvested from culture flasks by exposure to 0.25% trypsin/EDTA for 5 min at 37° C. Detached cells were collected, centrifuged (1500 rpm, 5 min, 4° C.) and rinsed to remove the trypsin solution. Cells were re-suspended in PBS without magnesium or calcium and counted. Cells were spun as described previously to remove PBS and a single cell suspension was created in 50% Matrigel: 50% PBS (v:v) so that a 100 μL subcutaneous injection would deliver 1.75 million cells per mouse. Tumors were established (˜150-250 mm³) in approximately 4 weeks post-injection.

### Drug Formulation

Compounds were administered in vehicles as described below.

| Compound | Vehicle |
|---|---|
| Compound A | 0.5% HPMC/0.1% TWEEN 80 pH 2.4 |
| Compound B | 0.5% HPMC/0.2% TWEEN 80 pH 7-8 |

### Experimental Protocol(s)

### Efficacy, Dosing and Measurement of Xenografts Tumors

Mice with similar sized tumors (100-300 mm³) were identified. The length and width of the tumors were measured by handheld calipers and body weights of the mice were measured using a bench top weighing scale. Tumor volumes were calculated based on the following formula: tumor volume=(Length×Width²)/2. Mice were block randomized into the required number of dosing groups (n=8 mice/group). The randomization process ensured that the average tumor volume for each dosing group was approximately equal at the beginning of the study and mice with differing tumor volumes were evenly distributed. Mice were placed in groups of eight accordingly and dosed with either vehicle or compound for 36 days either once or twice daily, as summarised in the table below. Mice were weighed and tumors measured twice weekly for the duration of the study using Studylog animal study management software (Studylog Systems, Inc., South San Francisco, Calif., USA).

| Group | Treatment |
|---|---|
| 1 | Vehicle |
| 2 | Compound A, 100 mg/kg, PO, BID |
| 3 | Compound B, 0.1 mg/kg, PO, QD |
| 4 | Compound B, 0.3 mg/kg, PO, QD + |
| | Compound A, 100 mg/kg, PO, BID |
| 5 | Compound B, 0.1 mg/kg, PO, QD + |
| | Compound A, 100 mg/kg, PO, BID |
| 6 | Compound B, 0.3 mg/kg, PO, QD |

### Data Analysis

Tumor volumes were calculated based on the following formula: tumor volume=(Length×Width²)/2. Tumor growth curves were created from the mean±standard error of the mean, with n=8 animals per group. The percentage of tumor growth inhibition was calculated on the final day of dosing using the following formula: 1−(tumor volume in drug-treated population/tumor volume in vehicle-treated control population).

### Results

Nude mice bearing A375P F11s tumor xenografts treated with vehicle alone showed a robust increase in tumor volume over the treatment duration reaching 1253 mm³ by day 28 of treatment. Compound A treatment at 100 mg/kg, BID resulted in 73.7% smaller tumor volume after 4 weeks of treatment, compared to vehicle treated animals. Mice treated with Compound B, at 0.3 mg/kg, QD, resulted in 70.4% smaller tumors compared to vehicle control; however the tumor growth at 0.1 mg/kg dose was similar to vehicle control. Mice treated with Compound A and 0.3 mg/kg of Compound B had tumor volumes which were 86.3% smaller than the vehicle treated animals on day 28 of treatment. Treatment was continued until day 37 in most of the drug-treated groups. Extended treatment showed a clear difference between the single agent activity of Compound B at 0.3 mg/kg and the same dose combined with Compound A. In the Compound B alone group, tumor volume started to increase even under continued drug treatment, whereas there was no increase in tumor volume when Compound B was given in combination with Compound A (Table 1-Table 2).

There was no significant effect on the body weight of mice treated with any of the agents (alone or in combination) in this study (Table 3).

TABLE 1
Mean A375P F11s Tumor Volume for different treatment groups on
various days during treatment

| Group | Treatment |
|---|---|
| 1 | Vehicle |
| 2 | Compound A, 100 mg/kg, PO, BID |
| 3 | Compound B, 0.1 mg/kg, PO, QD |
| 4 | Compound B, 0.3 mg/kg, PO, QD + |
| | Compound A, 100 mg/kg, PO, BID |
| 5 | Compound B, 0.1 mg/kg, PO, QD + |
| | Compound A, 100 mg/kg, PO, BID |
| 6 | Compound B, 0.3 mg/kg, PO, QD |

| | Study Day | | | | | | | | | | | |
|---|---|---|---|---|---|---|---|---|---|---|---|---|
| Group | 0 | 3 | 7 | 9 | 15 | 18 | 22 | 25 | 28 | 31 | 35 | 37 |
| 1 | 172.3 | 250.3 | 310.5 | 390.6 | 640.1 | 727.2 | 936.7 | 1,089.2 | 1,253.2 | ND | ND | ND |
| 2 | 158.1 | 187.1 | 222.7 | 257.6 | 252.1 | 308.6 | 273.3 | 306.3 | 329.9 | 346.5 | 334.7 | 381.8 |
| 3 | 199.3 | 260.5 | 300.8 | 335.1 | 459.3 | 616.2 | 916.9 | 1,100.3 | 1,338.4 | ND | ND | ND |
| 4 | 175.8 | 173.3 | 114.7 | 128.3 | 97.6 | 143.9 | 157.7 | 164.8 | 171.6 | 189.9 | 211.1 | 207.0 |
| 5 | 170.1 | 189.4 | 240.1 | 229.9 | 210.4 | 246.4 | 273.0 | 264.7 | 272.6 | 289.5 | 309.4 | 316.2 |
| 6 | 198.8 | 205.9 | 141.2 | 124.4 | 115.1 | 141.4 | 217.3 | 300.3 | 370.6 | 450.5 | 529.1 | 604.4 |

Mean tumor volume (mm³) on different days from start of treatment.
ND = Not determined (animals removed from study).

TABLE 2
Percent Inhibition of A375P F11s tumor volume treated with various agents
compared to vehicle treated mice

| Group | Treatment |
|---|---|
| 2 | Compound A, 100 mg/kg, PO, BID |
| 3 | Compound B, 0.1 mg/kg, PO, QD |
| 4 | Compound B, 0.3 mg/kg, PO, QD + |
| | Compound A, 100 mg/kg, PO, BID |
| 5 | Compound B, 0.1 mg/kg, PO, QD + |
| | Compound A, 100 mg/kg, PO, BID |
| 6 | Compound B, 0.3 mg/kg, PO, QD |

| | Study Day | | | | | | | | |
|---|---|---|---|---|---|---|---|---|---|
| Group | 0 | 3 | 7 | 9 | 15 | 18 | 22 | 25 | 28 |
| 2 | 8.2% | 25.3% | 28.3% | 34.1% | 60.6% | 57.6% | 70.8% | 71.9% | 73.7% |
| 3 | −15.70% | −4.1% | 3.1% | 14.2% | 28.2% | 15.3% | 2.1% | −1.0% | −6.8% |
| 4 | −2.0% | 30.8% | 63.0% | 67.2% | 84.8% | 80.2% | 83.2% | 84.9% | 86.3% |
| 5 | 1.3% | 24.3% | 22.7% | 41.1% | 67.1% | 66.1% | 70.9% | 75.7% | 78.2% |
| 6 | −15.41% | 17.7% | 54.5% | 68.1% | 82.0% | 80.5% | 76.8% | 72.4% | 70.4% |

Percent inhibition of tumor volume on different days from start of treatment.

TABLE 3
Mean body weight of nude mice treated with various agents either alone or in combination

| Group | Treatment |
|---|---|
| 1 | Vehicle |
| 2 | Compound A, 100 mg/kg, PO, BID |
| 3 | Compound B, 0.1 mg/kg, PO, QD |
| 4 | Compound B, 0.3 mg/kg, PO, QD + |
| | Compound A, 100 mg/kg, PO, BID |
| 5 | Compound B, 0.1 mg/kg, PO, QD + |
| | Compound A, 100 mg/kg, PO, BID |
| 6 | Compound B, 0.3 mg/kg, PO, QD |

| | Study Day | | | | | | | | | | | |
|---|---|---|---|---|---|---|---|---|---|---|---|---|
| Group | 0 | 3 | 7 | 9 | 15 | 18 | 22 | 25 | 28 | 31 | 35 | 37 |
| 1 | 25.33 | 25.59 | 26.43 | 26.59 | 26.77 | 27.36 | 27.26 | 27.33 | 27.45 | ND | ND | ND |
| 2 | 24.98 | 25.13 | 25.63 | 25.29 | 24.83 | 25.35 | 24.71 | 24.79 | 24.91 | 25.33 | 24.95 | 25.00 |
| 3 | 26.30 | 26.00 | 26.24 | 26.23 | 26.09 | 27.09 | 26.73 | 26.80 | 26.93 | ND | ND | ND |
| 4 | 25.33 | 24.66 | 24.58 | 24.55 | 23.98 | 24.84 | 24.28 | 24.23 | 24.25 | 24.46 | 24.41 | 24.65 |
| 5 | 25.44 | 25.28 | 24.96 | 24.90 | 24.60 | 25.15 | 24.41 | 24.41 | 24.29 | 24.55 | 24.73 | 24.94 |
| 6 | 24.59 | 24.10 | 23.89 | 23.93 | 23.94 | 24.41 | 23.83 | 23.95 | 24.18 | 24.49 | 24.54 | 24.64 |

Mean body weight (g) on different days from start of treatment (n = 8 mice/group).
ND = Not determined (animals removed from study).

Because the combinations of the present invention are active in the above assays they exhibit advantageous therapeutic utility in treating cancer.

Suitably, the present invention relates to a method for treating or lessening the severity of a cancer selected from: brain (gliomas), glioblastomas, astrocytomas, glioblastoma multiforme, Bannayan-Zonana syndrome, Cowden disease, Lhermitte-Duclos disease, breast, inflammatory breast cancer, Wilm's tumor, Ewing's sarcoma, Rhabdomyosarcoma, ependymoma, medulloblastoma, colon, head and neck, kidney, lung, liver, melanoma, ovarian, pancreatic, prostate, sarcoma, osteosarcoma, giant cell tumor of bone, thyroid,

Lymphoblastic T cell leukemia, Chronic myelogenous leukemia, Chronic lymphocytic leukemia, Hairy-cell leukemia, acute lymphoblastic leukemia, acute myelogenous leukemia, Chronic neutrophilic leukemia, Acute lymphoblastic T cell leukemia, Plasmacytoma, Immunoblastic large cell leukemia, Mantle cell leukemia, Multiple myeloma Megakaryoblastic leukemia, multiple myeloma, acute megakaryocytic leukemia, promyelocytic leukemia, Erythroleukemia,

malignant lymphoma, hodgkins lymphoma, non-hodgkins lymphoma, lymphoblastic T cell lymphoma, Burkitt's lymphoma, follicular lymphoma,

neuroblastoma, bladder cancer, urothelial cancer, lung cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor) and testicular cancer.

Suitably, the present invention relates to a method for treating or lessening the severity of a cancer selected from: brain (gliomas), glioblastomas, astrocytomas, glioblastoma multiforme, Bannayan-Zonana syndrome, Cowden disease, Lhermitte-Duclos disease, breast, colon, head and neck, kidney, lung, liver, melanoma, ovarian, pancreatic, prostate, sarcoma and thyroid.

Suitably, the present invention relates to a method for treating or lessening the severity of a cancer selected from ovarian, breast, pancreatic and prostate.

Suitably, the present invention relates to a method of treating or lessening the severity of a cancer that is either wild type or mutant for BRAF, KRAS, NRAS, HRAS, SOS1, NF1, or with activated receptor tyrosine kinases (e.g., EGFR, ErbB2, c-Kit, PDGFR, etc.). This includes patients who are wild type for each of, mutant for each of, and combinations of wild type and mutant of BRAF, KRAS, NRAS, HRAS, SOS1, NF1, and receptor tyrosine kinases (e.g., EGFR, ErbB2, c-Kit, PDGFR, etc.). The present invention also relates to a method of treating or lessening the severity of a cancer that has activated BRAF, KRAS, NRAS, HRAS, SOS1, NF1, or activated receptor tyrosine kinases (e.g., EGFR, ErbB2, c-Kit, PDGFR, etc.).e.g., by mutation or amplification of the gene or overexpression of the protein.

The term “wild type” as is understood in the art refers to a polypeptide or polynucleotide sequence that occurs in a native population without genetic modification. As is also understood in the art, a “mutant” includes a polypeptide or polynucleotide sequence having at least one modification to an amino acid or nucleic acid compared to the corresponding amino acid or nucleic acid found in a wild type polypeptide or polynucleotide, respectively. Included in the term mutant is Single Nucleotide Polymorphism (SNP) where a single base pair distinction exists in the sequence of a nucleic acid strand compared to the most prevalently found (wild type) nucleic acid strand.

Cancers that are either wild type or mutant for BRAF, KRAS, NRAS, HRAS, SOS1, NF1, EGFR, ErbB2, c-Kit, or PDGFR, or have amplification or overexpression of BRAF, KRAS, NRAS, HRAS, NF1, EGFR, ErbB2, c-Kit, or PDGFR, are identified by known methods.

For example, wild type or mutant BRAF, KRAS, NRAS, HRAS, SOS1, NF1, EGFR, ErbB2, c-Kit, or PDGFR, tumor cells can be identified by DNA amplification and sequencing techniques, DNA and RNA detection techniques, including, but not limited to Northern and Southern blot, respectively, and/or various biochip and array technologies or in-situ hybridization. Wild type and mutant polypeptides can be detected by a variety of techniques including, but not limited to immunodiagnostic techniques such as ELISA, Western blot or immunocytochemistry.

This invention provides a combination comprising 5-[[4-[(2,3-dimethyl-2H-indazol-6-yl)methylamino]-2-pyrimidinyl]amino]-2-methylbenzenesulfonamide, or a pharmaceutically acceptable salt, suitably the monohydrochloride salt, thereof, and N-{3-[3-cyclopropyl-5-(2-fluoro-4-iodo-phenylamino)6,8-dimethy;-2,4,7-trioxo-3,4,6,7-tetrahydro-2H-pyrido[4,3-d]pyrimidin-1-yl]phenyl}acetamide, or a pharmaceutically acceptable salt or solvate thereof, suitably the dimethyl sulfoxide solvate thereof.

This invention also provides for a combination comprising 5-[[4-[(2,3-dimethyl-2H-indazol-6-yl)methylamino]-2-pyrimidinyl]amino]-2-methylbenzenesulfonamide, or a pharmaceutically acceptable salt, suitably the monohydrochloride salt, thereof, and N-{3-[3-cyclopropyl-5-(2-fluoro-4-iodo-phenylamino)6,8-dimethy;-2,4,7-trioxo-3,4,6,7-tetrahydro-2H-pyrido[4,3-d]pyrimidin-1-yl]phenyl}acetamide, or a pharmaceutically acceptable salt or solvate thereof, suitably the dimethyl sulfoxide solvate thereof, for use in therapy.

This invention also provides for a combination comprising 5-[[4-[(2,3-dimethyl-2H-indazol-6-yl)methylamino]-2-pyrimidinyl]amino]-2-methylbenzenesulfonamide, or a pharmaceutically acceptable salt, suitably the monohydrochloride salt, thereof, and N-{3-[3-cyclopropyl-5-(2-fluoro-4-iodo-phenylamino)6,8-dimethy;-2,4,7-trioxo-3,4,6,7-tetrahydro-2H-pyrido[4,3-d]pyrimidin-1-yl]phenyl}acetamide, or a pharmaceutically acceptable salt or solvate thereof, suitably the dimethyl sulfoxide solvate thereof, for use in treating cancer.

This invention also provides a pharmaceutical composition comprising a combination of 5-[[4-[(2,3-dimethyl-2H-indazol-6-yl)methylamino]-2-pyrimidinyl]amino]-2-methylbenzenesulfonamide, or a pharmaceutically acceptable salt, suitably the monohydrochloride salt, thereof, and N-{3-[3-cyclopropyl-5-(2-fluoro-4-iodo-phenylamino)6,8-dimethy;-2,4,7-trioxo-3,4,6,7-tetrahydro-2H-pyrido[4,3-d]pyrimidin-1-yl]phenyl}acetamide, or a pharmaceutically acceptable salt or solvate thereof, suitably the dimethyl sulfoxide solvate thereof.

This invention also provides a combination kit comprising 5-[[4-[(2,3-dimethyl-2H-indazol-6-yl)methylamino]-2-pyrimidinyl]amino]-2-methylbenzenesulfonamide, or a pharmaceutically acceptable salt, suitably the monohydrochloride salt, thereof, and N-{3-[3-cyclopropyl-5-(2-fluoro-4-iodo-phenylamino)6,8-dimethy;-2,4,7-trioxo-3,4,6,7-tetrahydro-2H-pyrido[4,3-d]pyrimidin-1-yl]phenyl}acetamide, or a pharmaceutically acceptable salt or solvate thereof, suitably the dimethyl sulfoxide solvate thereof.

This invention also provides for the use of a combination comprising 5-[[4-[(2,3-dimethyl-2H-indazol-6-yl)methylamino]-2-pyrimidinyl]amino]-2-methylbenzenesulfonamide, or a pharmaceutically acceptable salt, suitably the monohydrochloride salt, thereof, and N-{3-[3-cyclopropyl-5-(2-fluoro-4-iodo-phenylamino)6,8-dimethy;-2,4,7-trioxo-3,4,6,7-tetrahydro-2H-pyrido[4,3-d]pyrimidin-1-yl]phenyl}acetamide, or a pharmaceutically acceptable salt or solvate thereof, suitably the dimethyl sulfoxide solvate thereof, in the manufacture of a medicament.

This invention also provides for the use of a combination comprising 5-[[4-[(2,3-dimethyl-2H-indazol-6-yl)methylamino]-2-pyrimidinyl]amino]-2-methylbenzenesulfonamide, or a pharmaceutically acceptable salt, suitably the monohydrochloride salt, thereof, and N-{3-[3-cyclopropyl-5-(2-fluoro-4-iodo-phenylamino)6,8-dimethy;-2,4,7-trioxo-3,4,6,7-tetrahydro-2H-pyrido[4,3-d]pyrimidin-1-yl]phenyl}acetamide, or a pharmaceutically acceptable salt or solvate thereof, suitably the dimethyl sulfoxide solvate thereof, in the manufacture of a medicament to treat cancer.

This invention also provides a method of treating cancer which comprises administering a combination of 5-[[4-[(2,3-dimethyl-2H-indazol-6-yl)methylamino]-2-pyrimidinyl]amino]-2-methylbenzenesulfonamide, or a pharmaceutically acceptable salt, suitably the monohydrochloride salt, thereof, and N-{3-[3-cyclopropyl-5-(2-fluoro-4-iodo-phenylamino)6,8-dimethy;-2,4,7-trioxo-3,4,6,7-tetrahydro-2H-pyrido[4,3-d]pyrimidin-1-yl]phenyl}acetamide, or a pharmaceutically acceptable salt or solvate thereof, suitably the dimethyl sulfoxide solvate thereof, to a subject in need thereof.

The following examples are intended for illustration only and are not intended to limit the scope of the invention in any way.

### Experimental Details

### EXAMPLE 1

### Capsule Composition

An oral dosage form for administering a combination of the present invention is produced by filing a standard two piece hard gelatin capsule with the ingredients in the proportions shown in Table I, below.

TABLE I

| INGREDIENTS | AMOUNTS |
|---|---|
| 5-[[4-[(2,3-dimethyl-2H-indazol-6-yl)methylamino]-2- | 200 mg |
| pyrimidinyl]amino]-2-methylbenzenesulfonamide | |
| hydrochloride (the monohydrochloride salt of Compound | |
| A) | |
| N-{3-[3-cyclopropyl-5-(2-fluoro-4-iodo-phenylamino)6,8- | 5 mg |
| dimethy; -2,4,7-trioxo-3,4,6,7-tetrahydro-2H-pyrido[4,3- | |
| d]pyrimidin-1-yl]phenyl}acetamide dimethyl sulfoxide | |
| (the dimethyl sulfoxide solvate of Compound B) | |
| Mannitol | 250 mg |
| Talc | 125 mg |
| Magnesium Stearate | 8 mg |

### EXAMPLE 2

### Capsule Composition

An oral dosage form for administering one of the compounds of the present invention is produced by filing a standard two piece hard gelatin capsule with the ingredients in the proportions shown in Table II, below.

TABLE II

| INGREDIENTS | AMOUNTS |
|---|---|
| 5-[[4-[(2,3-dimethyl-2H-indazol-6-yl)methylamino]-2- | 200 mg |
| pyrimidinyl]amino]-2-methylbenzenesulfonamide | |
| hydrochloride (the monohydrochloride salt of Compound | |
| A) | |
| Mannitol | 150 mg |
| Talc | 16 mg |
| Magnesium Stearate | 4 mg |

### EXAMPLE 3

### Capsule Composition

An oral dosage form for administering one of the compounds of the present invention is produced by filing a standard two piece hard gelatin capsule with the ingredients in the proportions shown in Table Ill, below.

TABLE III

| INGREDIENTS | AMOUNTS |
|---|---|
| N-{3-[3-cyclopropyl-5-(2-fluoro-4-iodo-phenylamino)6,8- | 5 mg |
| dimethy; -2,4,7-trioxo-3,4,6,7-tetrahydro-2H-pyrido[4,3- | |
| d]pyrimidin-1-yl]phenyl}acetamide dimethyl sulfoxide | |
| (the dimethyl sulfoxide solvate of Compound B) | |
| Mannitol | 150 mg |
| Talc | 12 mg |
| Magnesium Stearate | 8 mg |

### EXAMPLE 4

### Tablet Composition

The sucrose, microcrystalline cellulose and the compounds of the invented combination, as shown in Table IV below, are mixed and granulated in the proportions shown with a 10% gelatin solution. The wet granules are screened, dried, mixed with the starch, talc and stearic acid, then screened and compressed into a tablet.

TABLE IV

| INGREDIENTS | AMOUNTS |
|---|---|
| 5-[[4-[(2,3-dimethyl-2H-indazol-6-yl)methylamino]-2- | 200 mg |
| pyrimidinyl]amino]-2-methylbenzenesulfonamide | |
| hydrochloride (the monohydrochloride salt of Compound | |
| A) | |
| N-{3-[3-cyclopropyl-5-(2-fluoro-4-iodo-phenylamino)6,8- | 5 mg |
| dimethy; -2,4,7-trioxo-3,4,6,7-tetrahydro-2H-pyrido[4,3- | |
| d]pyrimidin-1-yl]phenyl}acetamide dimethyl sulfoxide | |
| (the dimethyl sulfoxide solvate of Compound B) | |
| Microcrystalline cellulose | 300 mg |
| sucrose | 10 mg |
| starch | 40 mg |
| talc | 20 mg |
| stearic acid | 5 mg |

### EXAMPLE 5

### Tablet Composition

The sucrose, microcrystalline cellulose and one of the compounds of the invented combination, as shown in Table V below, are mixed and granulated in the proportions shown with a 10% gelatin solution. The wet granules are screened, dried, mixed with the starch, talc and stearic acid, then screened and compressed into a tablet.

TABLE V

| INGREDIENTS | AMOUNTS |
|---|---|
| 5-[[4-[(2,3-dimethyl-2H-indazol-6-yl)methylamino]-2- | 200 mg |
| pyrimidinyl]amino]-2-methylbenzenesulfonamide | |
| hydrochloride (the monohydrochloride salt of Compound | |
| A) | |
| Microcrystalline cellulose | 200 mg |
| sucrose | 4 mg |
| starch | 2 mg |
| talc | 1 mg |
| stearic acid | 0.5 mg |

### EXAMPLE 6

### Tablet Composition

The sucrose, microcrystalline cellulose and one of the compounds of the invented combination, as shown in Table VI below, are mixed and granulated in the proportions shown with a 10% gelatin solution. The wet granules are screened, dried, mixed with the starch, talc and stearic acid, then screened and compressed into a tablet.

TABLE VI

| INGREDIENTS | AMOUNTS |
|---|---|
| N-{3-[3-cyclopropyl-5-(2-fluoro-4-iodo-phenylamino)6,8- | 5 mg |
| dimethy; -2,4,7-trioxo-3,4,6,7-tetrahydro-2H-pyrido[4,3- | |
| d]pyrimidin-1-yl]phenyl}acetamide dimethyl sulfoxide | |
| (the dimethyl sulfoxide solvate of Compound B) | |
| Microcrystalline cellulose | 300 mg |
| sucrose | 40 mg |
| starch | 20 mg |
| talc | 10 mg |
| stearic acid | 5 mg |

While the preferred embodiments of the invention are illustrated by the above, it is to be understood that the invention is not limited to the precise instructions herein disclosed and that the right to all modifications coming within the scope of the following claims is reserved.

We claim:

1. A combination comprising:
(i) a compound of Structure (I): 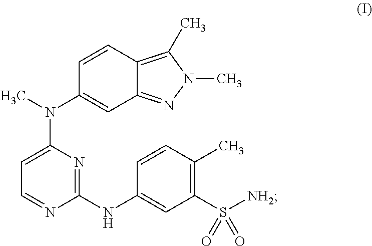 [SMILES: Cc1ccc(Nc2nccc(N(C)c3ccc4c(C)n(C)nc4c3)n2)cc1S(N)(=O)=O]
or a pharmaceutically acceptable salt thereof; and
(ii) a compound of Structure (II): 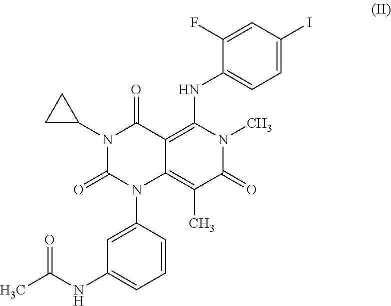 [SMILES: CC(=O)Nc1cccc(-n2c(=O)n(C3CC3)c(=O)c3c(Nc4ccc(I)cc4F)n(C)c(=O)c(C)c32)c1]
or a pharmaceutically acceptable salt or solvate thereof.

2. A combination according to claim 1 where the compound of Structure (I) is in the form of a monohydrochloride salt and the compound of Structure (II) is in the form of a dimethyl sulfoxide solvate.

3. A combination kit comprising a combination according to claim 1 together with a pharmaceutically acceptable carrier or carriers.

4. A combination according to claim 2 where the amount of the compound of Structure (I) is an amount selected from 50 mg to 1,200 mg, and that amount is administered once per day in one or more tablets, and the amount of the compound of Structure (II) is an amount selected from 0.125 mg to 10 mg, and that amount is administered once per day.

5. A method of treating cancer in a human in need thereof which comprises the in vivo administration of a therapeutically effective amount of a combination of 5-[[4-[(2,3-dimethyl-2H-indazol-6-yl)methylamino]-2-pyrimidinyl]amino]-2-methylbenzenesulfonamide, or a pharmaceutically acceptable salt thereof, and N-{3-[3-cyclopropyl-5-(2-fluoro-4-iodo-phenylamino)6,8-dimethy;-2,4,7-trioxo-3,4,6,7-tetrahydro-2H-pyrido[4,3-d]pyrimidin-1-yl]phenyl}acetamide, or a pharmaceutically acceptable salt or solvate thereof, to such human,
wherein the combination is administered within a specified period, and
wherein the combination is administered for a duration of time.

6. A method according to claim 5 wherein the amount of 5-[[4-[(2,3-dimethyl-2H-indazol-6-yl)methylamino]-2-pyrimidinyl]amino]-2-methylbenzenesulfonamide, or a pharmaceutically acceptable salt thereof, is selected from about 100 mg to about 1,000 mg, and that amount is administered once per day in one or more tablets, and the amount of N-{3-[3-cyclopropyl-5-(2-fluoro-4-iodo-phenylamino)6,8-dimethy;-2,4,7-trioxo-3,4,6,7-tetrahydro-2H-pyrido[4,3-d]pyrimidin-1-yl]phenyl}acetamide, or a pharmaceutically acceptable salt of solvate thereof, is selected from about 0.125 mg to about 10 mg, and that amount is administered once per day.

7. A method according to claim 6 wherein the amount of 5-[[4-[(2,3-dimethyl-2H-indazol-6-yl)methylamino]-2-pyrimidinyl]amino]-2-methylbenzenesulfonamide, or a pharmaceutically acceptable salt thereof, is selected from about 100 mg to about 800 mg, and that amount is administered once per day in one or more tablets, and the amount of N-{3-[3-cyclopropyl-5-(2-fluoro-4-iodo-phenylamino)6,8-dimethy;-2,4,7-trioxo-3,4,6,7-tetrahydro-2H-pyrido[4,3-d]pyrimidin-1-yl]phenyl}acetamide dimethyl sulfoxide is selected from about 1 mg to about 9 mg, and that amount is administered once per day.

8. A method according to claim 7 wherein 5-[[4-[(2,3-dimethyl-2H-indazol-6-yl)methylamino]-2-pyrimidinyl]amino]-2-methylbenzenesulfonamide monohydrochloride and N-{3-[3-cyclopropyl-5-(2-fluoro-4-iodo-phenylamino)6,8-dimethy;-2,4,7-trioxo-3,4,6,7-tetrahydro-2H-pyrido[4,3-d]pyrimidin-1-yl]phenyl}acetamide dimethyl sulfoxide, are administered within 12 hours of each other for from 1 to 3 consecutive days followed by administration of 5-[[4-[(2,3-dimethyl-2H-indazol-6-yl)methylamino]-2-pyrimidinyl]amino]-2-methylbenzenesulfonamide monohydrochloride for from 3 to 7 consecutive days, optionally followed by one or more cycles of repeat dosing.

9. A method according to claim 5 wherein the cancer is selected from: brain (gliomas), glioblastomas, astrocytomas, glioblastoma multiforme, Bannayan-Zonana syndrome, Cowden disease, Lhermitte-Duclos disease, breast, inflammatory breast cancer, Wilm's tumor, Ewing's sarcoma, Rhabdomyosarcoma, ependymoma, medulloblastoma, colon, head and neck, kidney, lung, liver, melanoma, ovarian, pancreatic, prostate, sarcoma, osteosarcoma, giant cell tumor of bone, thyroid,
Lymphoblastic T cell leukemia, Chronic myelogenous leukemia, Chronic lymphocytic leukemia, Hairy-cell leukemia, acute lymphoblastic leukemia, acute myelogenous leukemia, Chronic neutrophilic leukemia, Acute lymphoblastic T cell leukemia, Plasmacytoma, Immunoblastic large cell leukemia, Mantle cell leukemia, Multiple myeloma Megakaryoblastic leukemia, multiple myeloma, acute megakaryocytic leukemia, promyelocytic leukemia, Erythroleukemia,
malignant lymphoma, hodgkins lymphoma, non-hodgkins lymphoma, lymphoblastic T cell lymphoma, Burkitt's lymphoma, follicular lymphoma,
neuroblastoma, bladder cancer, urothelial cancer, lung cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor) and testicular cancer.

10. A method according to claim 9 wherein the amount of 5-[[4-[(2,3-dimethyl-2H-indazol-6-yl)methylamino]-2-pyrimidinyl]amino]-2-methylbenzenesulfonamide, or a pharmaceutically acceptable salt thereof, is selected from about 100 mg to about 1,000 mg, and that amount is administered once per day in one or more tablets, and the amount of N-{3-[3-cyclopropyl-5-(2-fluoro-4-iodo-phenylamino)6,8-dimethy;-2,4,7-trioxo-3,4,6,7-tetrahydro-2H-pyrido[4,3-d]pyrimidin-1-yl]phenyl}acetamide, or a pharmaceutically acceptable salt or solvate thereof, is selected from about 0.125 mg to about 10 mg, and that amount is administered once per day.

11. A method according to claim 10 wherein the amount of 5-[[4-[(2,3-dimethyl-2H-indazol-6-yl)methylamino]-2-pyrimidinyl]amino]-2-methylbenzenesulfonamide, or a pharmaceutically acceptable salt thereof, is selected from about 100 mg to about 800 mg, and that amount is administered once per day in one or more tablets, and the amount of N-{3-[3-cyclopropyl-5-(2-fluoro-4-iodo-phenylamino)6,8-dimethy;-2,4,7-trioxo-3,4,6,7-tetrahydro-2H-pyrido[4,3-d]pyrimidin-1-yl]phenyl}acetamide, or a pharmaceutically acceptable salt or solvate thereof, is selected from about 1 mg to about 9 mg, and that amount is administered once per day.

12. A method according to claim 11 wherein 5-[[4-[(2,3-dimethyl-2H-indazol-6-yl)methylamino]-2-pyrimidinyl]amino]-2-methylbenzenesulfonamide monohydrochloride and N-{3-[3-cyclopropyl-5-(2-fluoro-4-iodo-phenylamino)6,8-dimethy;-2,4,7-trioxo-3,4,6,7-tetrahydro-2H-pyrido[4,3-d]pyrimidin-1-yl]phenyl}acetamide dimethyl sulfoxide, are administered within 12 hours of each other for at least 14 days.

13. A method according to claim 9 wherein the cancer selected from ovarian, breast, pancreatic and prostate.

14. A method according to claim 10 wherein the cancer selected from ovarian, breast, pancreatic and prostate.

15. A method according to claim 12 wherein the cancer selected from ovarian, breast, pancreatic and prostate.

16. A method treating a cancer selected from: brain (gliomas), glioblastomas, astrocytomas, glioblastoma multiforme, Bannayan-Zonana syndrome, Cowden disease, Lhermitte-Duclos disease, breast, inflammatory breast cancer, Wilm's tumor, Ewing's sarcoma, Rhabdomyosarcoma, ependymoma, medulloblastoma, colon, head and neck, kidney, lung, liver, melanoma, ovarian, pancreatic, prostate, sarcoma, osteosarcoma, giant cell tumor of bone, thyroid,
Lymphoblastic T cell leukemia, Chronic myelogenous leukemia, Chronic lymphocytic leukemia, Hairy-cell leukemia, acute lymphoblastic leukemia, acute myelogenous leukemia, Chronic neutrophilic leukemia, Acute lymphoblastic T cell leukemia, Plasmacytoma, Immunoblastic large cell leukemia, Mantle cell leukemia, Multiple myeloma Megakaryoblastic leukemia, multiple myeloma, acute megakaryocytic leukemia, promyelocytic leukemia, Erythroleukemia,
malignant lymphoma, hodgkins lymphoma, non-hodgkins lymphoma, lymphoblastic T cell lymphoma, Burkitt's lymphoma, follicular lymphoma,
neuroblastoma, bladder cancer, urothelial cancer, lung cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor) and testicular cancer;
in a human in need thereof which comprises the in vivo administration of a therapeutically effective amount of a combination of 5-[[4-[(2,3-dimethyl-2H-indazol-6-yl)methylamino]-2-pyrimidinyl]amino]-2-methylbenzenesulfonamide, or a pharmaceutically acceptable salt thereof, and N-{3-[3-cyclopropyl-5-(2-fluoro-4-iodo-phenylamino)6,8-dimethy;-2,4,7-trioxo-3,4,6,7-tetrahydro-2H-pyrido[4,3-d]pyrimidin-1-yl]phenyl}acetamide, or a pharmaceutically acceptable salt or solvate thereof, to such human,
wherein the compounds of the combination are administered sequentially.

17. A method according to claim 16 wherein the amount of 5-[[4-[(2,3-dimethyl-2 H-indazol-6-yl)methylamino]-2-pyrimidinyl]amino]-2-methylbenzenesulfonamide, or a pharmaceutically acceptable salt thereof, is selected from about 100 mg to about 1,000 mg, and that amount is administered once per day in one or more tablets, and the amount of N-{3-[3-cyclopropyl-5-(2-fluoro-4-iodo-phenylamino)6,8-dimethy;-2,4,7-trioxo-3,4,6,7-tetrahydro-2H-pyrido[4,3-d]pyrimidin-1-yl]phenyl}acetamide, or a pharmaceutically acceptable salt or solvate thereof, is selected from about 0.125 mg to about 10 mg, and that amount is administered once per day.

18. A method according to claim 17 wherein the amount of 5-[[4-[(2,3-dimethyl-2 H-indazol-6-yl)methylamino]-2-pyrimidinyl]amino]-2-methylbenzenesulfonamide, or a pharmaceutically acceptable salt thereof, is selected from about 100 mg to about 800 mg, and that amount is administered once per day in one or more tablets, and the amount of N-{3-[3-cyclopropyl-5-(2-fluoro-4-iodo-phenylamino)6,8-dimethy;-2,4,7-trioxo-3,4,6,7-tetrahydro-2H-pyrido[4,3-d]pyrimidin-1-yl]phenyl}acetamide, or a pharmaceutically acceptable salt or solvate thereof, is selected from about 1 mg to about 9 mg, and that amount is administered once per day.

19. A method according to claim 18 wherein 5-[[4-[(2,3-dimethyl-2H-indazol-6-yl)methylamino]-2-pyrimidinyl]amino]-2-methylbenzenesulfonamide monohydrochloride is administered for from 1 to 30 consecutive days, followed by an optional drug holiday of from 1 to 14 days, followed by administration of N-{3-[3-cyclopropyl-5-(2-fluoro-4-iodo-phenylamino)6,8-dimethy;-2,4,7-trioxo-3,4,6,7-tetrahydro-2H-pyrido[4,3-d]pyrimidin-1-yl]phenyl}acetamide dimethyl sulfoxide, for from 1 to 30 days, optionally followed by one or more cycles of repeat dosing.

20. A method according to claim 16 wherein the cancer selected from ovarian, breast, pancreatic and prostate.
